(19) United States
(12) Patent Application Publication
(10) Pub. No.: US 2011/0259774 A1
(43) Pub. Date: Oct. 27, 2011

(54) COVER WITH DEVICE ACTUATION FEATURE

(75) Inventor: Simon Rørvig, Frederiksberg (DK)

(73) Assignee: Novo Nordisk A/S, Bagsvaerd (DK)

(21) Appl. No.: 13/063,358

(22) PCT Filed: Sep. 10, 2009
(86) PCT No.: PCT/EP2009/061738

§ 371 Date: Jul. 13, 2011

(30) Foreign Application Priority Data
Sep. 10, 2008 (DK) PA 200801261

(51) Int. Cl. A61B 19/02; B23P 19/00

(52) U.S. Cl. 206/438; 29/428

(57) ABSTRACT

The invention provides an assembly comprising a cover and a device arranged fully or partly in the cover. The device comprises a first portion, and a second portion moveable relative to the first portion between an initial and an actuated position, and the cover comprises a cavity portion and a closure portion which together form an enclosure in which the device is arranged initially. Movement of the closure portion from an initial position in a first direction results in the second portion of the device being moved in a second direction between the initial and the actuated position, the first direction being different from the second direction. By this arrangement it is possible to provide a cover-device assembly which is easy and intuitive to use and open and at the same time ensures that the enclosed device is actuated correctly.

The present invention generally relates to a cover or a packaging for a device comprising a moveable structure, the cover or packaging being adapted to activate the moveable structure when opened by a user.

### BACKGROUND OF THE INVENTION

In the disclosure and discussion of the present invention reference is made to the treatment of diabetes by delivery of e.g. insulin, however, this is only an exemplary use of the present invention.

Portable drug delivery devices for delivering a drug to a patient are well known and generally comprise a reservoir adapted to contain a liquid drug and having an outlet in fluid communication with a transcutaneous access device such as a soft cannula or a hollow infusion needle, as well as expelling means for expelling a drug out of the reservoir and through the skin of the subject via the cannula or needle, the latter often in the form of an infusion set. Such devices are often termed infusion pumps.

Basically, infusion pumps can be divided into two classes. The first class comprises durable infusion pumps which are relatively expensive pumps intended for 3-4 years use, for which reason the initial cost for such a pump often is a barrier to this type of therapy. Although more complex than traditional syringes and pens, the pump offers the advantages of continuous infusion of insulin, precision in dosing and optionally e.g. programmable delivery profiles and user actuated bolus infusions in connections with meals.

Addressing the above problem, several attempts have been made to provide a second class of drug infusion devices that are low in cost and convenient to use. Some of these devices are intended to be partially or entirely disposable and may provide many of the advantages associated with an infusion pump without the attendant cost and inconveniences, e.g. the pump may be prefilled thus avoiding the need for filling or refilling a drug reservoir. Examples of this type of infusion devices are known from U.S. Pat. Nos. 4,340,048 and 4,552,561 (based on osmotic pumps), U.S. Pat. No. 5,858,001 (based on a piston pump), U.S. Pat. No. 6,280,148 (based on a membrane pump), U.S. Pat. No. 5,957,895 (based on a flow restrictor pump (also know as a bleeding hole pump), U.S. Pat. No. 5,527,288 (based on a gas generating pump), or U.S. Pat. No. 5,814,020 (based on a swellable gel) which all in the last decades have been proposed for use in inexpensive, primarily disposable drug infusion devices, the cited documents being incorporated by reference.

The disposable pumps generally comprises a skin-contacting mounting surface adapted for application to the skin of a subject by adhesive means, and with the infusion cannula or needle arranged such that in a situation of use it projects from the mounting surface to thereby penetrate the skin of the user, whereby the place where the needle penetrates the skin is covered while the appliance is in use. This kind of disposable pump are often referred to as a “patch pump” and may be either unitary as disclosed in U.S. Pat. No. 6,699,218, or in the form of an assembly comprising two or more components which when assembled constitutes a patch pump as disclosed in e.g. WO 2005/039673.

When a disposable device such as a pump is supplied to the user it normally has to be activated before being put into use. For example, the pump disclosed in U.S. Pat. No. 6,699,218 is supplied to the user with an empty reservoir which is then to be filled by the user before being mounted to a skin surface. The pump comprises electronic circuitry for communicating with a remote controller and for controlling the pump mechanism expelling fluid drug from the reservoir. In the disclosed embodiment the circuitry is activated from its dormant state when the reservoir is being filled, however, this could also be done by actuation of a user-actuatable switch.

The pump may also be supplied to the user with a pre-filled reservoir which then has to be connected to a fluid-conducting structure before being put into use. In the pre-filled pump assembly disclosed in US 2006/264835, hereby incorporated by reference, a pump-reservoir unit is provided in which a flexible, pre-filled and sealed reservoir is provided in combination with a down-stream electronically controlled suction pump. Before the unit can be taken into use the pump has to be connected with the reservoir and the electronics have to be activated. WO 2009/021950 shows a pump designed to be used in a pump-reservoir unit as disclosed in US 2006/264835, this pump being adapted to be arranged moveable relative to the reservoir, such that the pump has to be pushed towards the reservoir before the unit is activated and can be mounted on the patch unit. The actuation could be done manually by the user or it could happen when the pump-reservoir unit is mounted to the patch unit, however, it may be considered an advantage if the pump-reservoir unit was fully activated and ready-for-use when it was taken out of its primary packaging by the user.

Having regard to the above-identified problems, it is an object of the present invention to provide an arrangement which ensures that an assembly comprising a moveable structure can be activated when a packaging or cover fully or partly surrounding the assembly is opened by a user. In case the enclosed assembly is optimized for e.g. small size or low production costs the actuation motion may be “in conflict” with the intuitive way of handling the enclosing packaging material. Thus, the package or cover should be easy and intuitive to open but still ensure that the enclosed assembly is activated correctly and reliably. This said, the cost of the packaging and its assembly should still be cost-effective.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, corresponding to a first aspect, an assembly is provided comprising a device and a cover arranged to cover at least a portion of the device in an initial position. The device comprises a first portion, and a second portion moveable relative to the first portion between an initial and an actuated position, wherein movement of at least a portion of the cover from the initial position in a first direction results in the second portion of the device being moved in a second direction between the initial and the actuated position, the first direction being different from the second direction. The force required to move the second portion of the device in the second direction is provided fully or in part by movement of the at least a portion of the cover, i.e. energy necessary to move the second portion is provided by the user when moving the cover or a portion thereof. As appears, this is different from traditional “actuation designs” in which movement of e.g. a stop by the user will allow a biased part to move, e.g. driven by a pre-stressed spring as disclosed in WO 2004/041334. By the arrangement of the invention it is possible to provide a cover-device assembly which is simple in design, easy and intuitive to use and open and at the same time ensures that the covered device is actuated correctly.

In illustrative examples the first and second directions may be essentially opposite to each other, however, the first and second directions may be based on movement of the same or different “kinds”, e.g. axial, rotational or composite wherein movement of different kinds always can be considered in different directions, e.g. when a rotational movement is translated into an axial or vice versa.

In a first exemplary embodiment an assembly is provided comprising a removable cover in the form of a container and a device arranged in the container. The device comprises a first portion, and a second portion moveable relative to the first portion between an initial and an actuated position, and the container comprises a cavity portion and a closure member which together form an enclosure in which the device is arranged initially, wherein movement of the closure member from an initial position in a first direction results in the second portion of the device being moved in a second direction between the initial and the actuated position, the first direction being different from the second direction.

To accomplish the translation of movement the container may be provided with an actuation structure which moves the second portion of the device in the second direction, however, it could also be pulled or rotated. The actuation structure may comprise a pivot member with a proximal and a distal portion, wherein the pivot member initially is connected to either the cavity portion or the first portion of the device by a pivoting joint located between the proximal and the distal portion. The closure member is connected to the pivoting member in such a way that movement of the closure member from the initial position results in a pivoting movement of the distal portion of the pivot member, whereby the pivoting movement pushes, pulls or rotates the second portion of the device in the second direction.

In an exemplary embodiment the pivoting joint is adapted to disengage when the pivot member has moved the second portion of the device to its actuated position, this arrangement allowing the closure member to be removed from the cavity portion. The closure member may comprise an outer shell connected to the proximal portion of the pivot member by a flexible connection (e.g. hinged structure or string), this allowing a pulling force to be transmitted to the pivot member when the closure member is moved in the first direction.

The actuation structure may comprise two pivoting members arranged to pivot in opposite directions when the closure member is moved from the initial position, whereby the distal portions of the pivot members will be moved in opposite directions. In an exemplary embodiment the actuation structure is adapted to exert an essentially axial force on the second portion relative to a predetermined direction of movement between the first and second portion, this preventing kinking of the second portion relative to the first portion in respect of the predetermined direction of movement. A pair of pivot members being moved against or towards each other could be used to ensure such an axial force.

The first device portion may be in the form a housing with an opening in which the second device portion is slidably arranged, the closure member in its initial position sealing the opening. The container comprises locking means for directly or indirectly locking the second portion relative to the first portion when the cover is in its initial position, the locking means being adapted to unlock when the cover is moved away from its initial position. For example, the first portion may be locked to the closure member and the second portion to the cavity portion, the closure member and the cavity portion being locked to each other. The cover may be arranged to move a predetermined distance to unlock the locking means before the second portion of the device is being moved.

In accordance with the above-described first exemplary embodiment an assembly is provided in which a device is enclosed in a container from which it can be removed, however, the invention may also be realized in a configuration corresponding to a second exemplary embodiment in which an assembly is provided comprising a device and a cover attached to a portion thereof, where the device comprises a first portion, and a second portion moveable relative to the first portion between an initial and an actuated position. Movement and/or removal of the cover from an initial position in a first direction results in the second portion of the device being moved in a second direction between the initial and the actuated position, the first direction being different from the second direction. This configuration may then be modified corresponding to the above disclosure of the first exemplary embodiment.

More generally, and corresponding to a further aspect of the invention, an assembly is provided comprising a sub-assembly and a cover attached to a portion thereof, the sub-assembly comprising a first portion, and a second portion moveable relative to the first portion between an initial and an actuated position, wherein movement of the cover from an initial position in a first direction results in the second portion of the sub-assembly being moved in a second direction between the initial and the actuated position, the first direction being different from the second direction.

The sub-assembly may be in the form of a device comprising the first and second portion, with the cover being attached to the first portion. Alternatively, the sub-assembly may be in the form of a device arranged at least partly in the interior of a cavity portion, the cavity portion and the cover together forming an enclosure in which the device is arranged initially.

For all of the above embodiments of the invention, the first portion may comprise a fluid-filled reservoir (e.g. containing a fluid drug), and the second portion may comprise a fluid conducting structure, the fluid conducting structure being adapted to be arranged in fluid communication with the reservoir in the actuated position. The fluid conduction structure may be in the form of an electronically controlled suction pump, e.g. a membrane pump. As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject.

In a further aspect of the invention a method for arranging a device in a container is provided, comprising the steps of (a) providing a container comprising a cavity portion and a closure portion which together form an enclosure, (b) providing a device adapted to be arranged at least partly in the cavity portion, (c) providing an actuation unit comprising a pivot member, the actuation unit being adapted to lock to the closure portion, (d) arranging the device at least partly in the cavity portion, (e) arranging the actuation unit with the pivot member in pivoting engagement with either the cavity portion or the device, (f) arranging the closure portion in engagement with the cavity portion to thereby form an enclosure in which the device is arranged, and (g) locking the actuation unit to the cover portion. As appears, the described method allows the different components to be assembled efficiently in a “stacked” fashion.

The device may comprise a first portion and a second portion moveable relative to the first portion between an initial and an actuated position, and the actuation unit may be adapted to provide a pivoting movement of the pivot member when the cover portion is moved away from the cavity portion, the pivoting movement moving the second portion of the device towards the actuated position. In order to allow the cover portion to be removed from the cavity portion the pivot member may be adapted to be released from the pivoting engagement with the cavity portion or the device when the. The actuation unit may comprise a hinged structure adapted to be reversibly locked in an initial folded configuration, the actuation unit being arranged in engagement with either the cavity portion or the device in its folded configuration.

### BRIEF DESCRIPTION OF THE DRAWINGS

In the following the invention will be further described with reference to the drawings, wherein

FIG. 1 shows a pump unit,

FIG. 2 shows a case portion for a container adapted to receive the pump unit of FIG. 1,

FIG. 3 shows an un-folded actuation member,

FIG. 4 shows the actuation member of FIG. 3 in a folded configuration,

FIG. 5 shows a locking member,

FIG. 6 shows the actuation member and the case portion in an assembled state,

FIG. 7 shows the case portion with a cover portion mounted to form a container,

FIGS. 8-11 show how a device arranged in a container is actuated as a cover is removed from the container,

FIG. 12 shows a first alternative embodiment in which a cover is arranged on a device,

FIG. 13 shows a further alternative embodiment of a device arranged in a container, and

FIG. 14 shows a yet further alternative embodiment of a device arranged in a container.

In the figures like structures are mainly identified by like reference numerals.

### DESCRIPTION OF EXEMPLARY EMBODIMENTS

When in the following terms such as “upper” and “lower”, “right” and “left”, “above” and “below”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

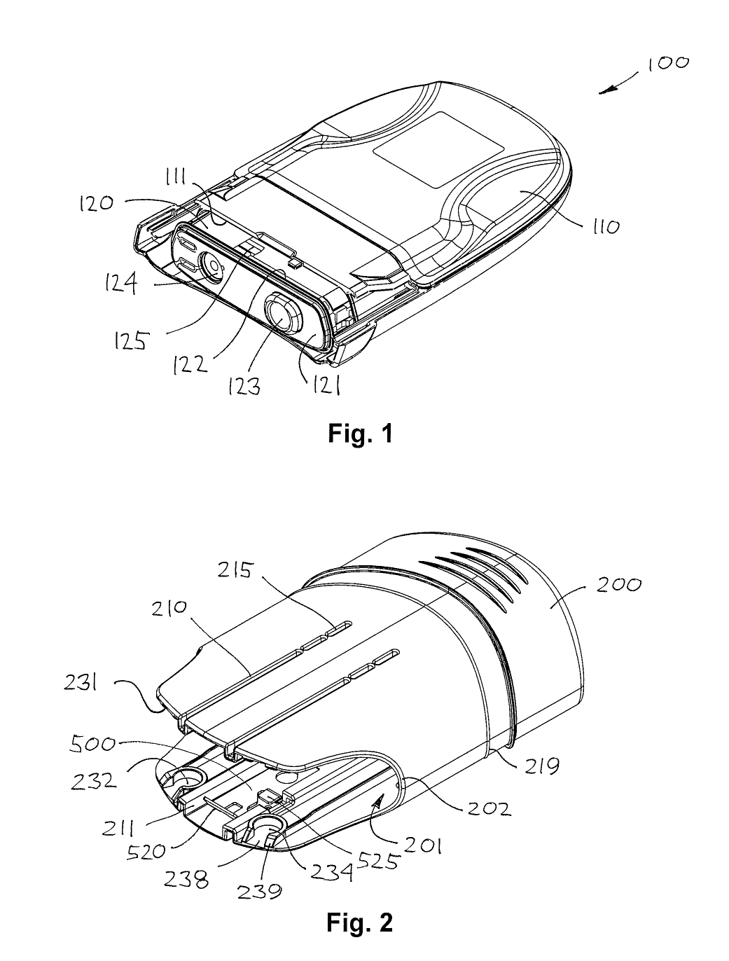

In the following an embodiment of the present invention will be described in which an actuatable pump unit is arranged in a packaging container from which it is to be removed. The pump unit 100 comprises a housing 110 in which a pump assembly 120 is slidingly mounted. The container 101 (see FIG. 7) comprises a case or cavity portion 200 (in the following “case”) provided with a locking member 500 and a cover portion or member 300 (in the following “cover”) provided with an actuation member 400, where the case and cover when assembled defines an enclosure for the pump unit. Before turning to a detailed description of how the pump unit will be actuated during opening of the container, the different functional components of the individual members of the embodiment will be described with reference to FIGS. 1-7.

FIG. 1 shows a pump unit 100 comprising a housing 110 with an opening 111 in which a pump assembly 120 is slidingly mounted in an initial position. In the housing are further arranged a drug-filled reservoir and electronic circuitry for controlling and driving the pump assembly. The pump assembly comprises a pump having a fluid inlet facing towards the interior of the housing and a fluid outlet septum 123, as well as a pump actuator with a contact 124 by which the pump can be started. During actuation the pump assembly is moved into the housing from an initial position to an actuated position in which fluid communication is established between the reservoir and the pump inlet, and electric contact is established between the circuitry and the pump actuator. When actuated the pump unit is adapted to be mounted to a corresponding skin-mountable patch unit, whereby the contact 124 is activated and a fluid communication between the pump outlet 123 and a fluid conducting structure (e.g. a cannula) of the patch unit is established, this allowing a fluid drug contained in the reservoir to be infused subcutaneously through the cannula. In the shown embodiment the pump assembly comprises a face plate 121 with a peripheral lip seal 122 adapted to engage the housing opening to thereby seal the interior of the pump unit in the actuated state. The pump assembly is further provided with an indentation 125 adapted to engage a locking structure of the container, this preventing dislocation of the pump assembly during transport. Further details about such a pump-patch device is given below, however, it should be emphasized that the present invention is not specifically related to a pump unit but can be used in combination with any device for which a motion-actuation is relevant and which has to be unpacked prior to use. In FIG. 1 the surface of the pump unit adapted to face the skin of the patient is shown facing upwards for which reason most of the figures of this application will show embodiments of the invention seen from “below”. The upper surface of the pump housing comprises a free edge portion 112 (see FIG. 11) which serves as locking means when the pump unit is attached to the patch unit.

FIG. 2 shows from “above” a case 200 having an interior 201 adapted to receive the pump unit 100. The case is provided with two pairs of exterior grooves 210 (forming corresponding interior ridges 211) adapted to engage corresponding guide flanges 301 of the cover, as well as a circumferential seal structure 219 adapted to engage an interior wall of the cover to thereby provide a sealed enclosure for the pump unit. The case is provided with a locking member 500 in the form of a flexible latch having a proximal portion attached to the case and a distal free portion, the latter comprising a latch 525 adapted to engage indentation 125 in the pump assembly as well as a slanted edge 520 adapted to engage projections 411, 412 of the actuation member 400. The case is further provided with a first pair of opposed axle seats 231, 232 and a second pair of opposed axle seats 233, 234, thereby allowing two elements of the actuation member 400 to be mounted in pivoting engagement. The two pairs of axle seats are arranged symmetrically relative to a central plane of the case (as represented by the “cutting plane” in the drawings). As appears, the seats each has a peripheral opening 238, this allowing the axle heads to disengage from the seats when correspondingly positioned, see below. Each opening is flanked by a pair of ramps 239 allowing an axle head to be inserted into the seat, see below. To allow a user to grip the pump unit the after the cover has been removed, the case comprises two lateral cut-out portions 202.

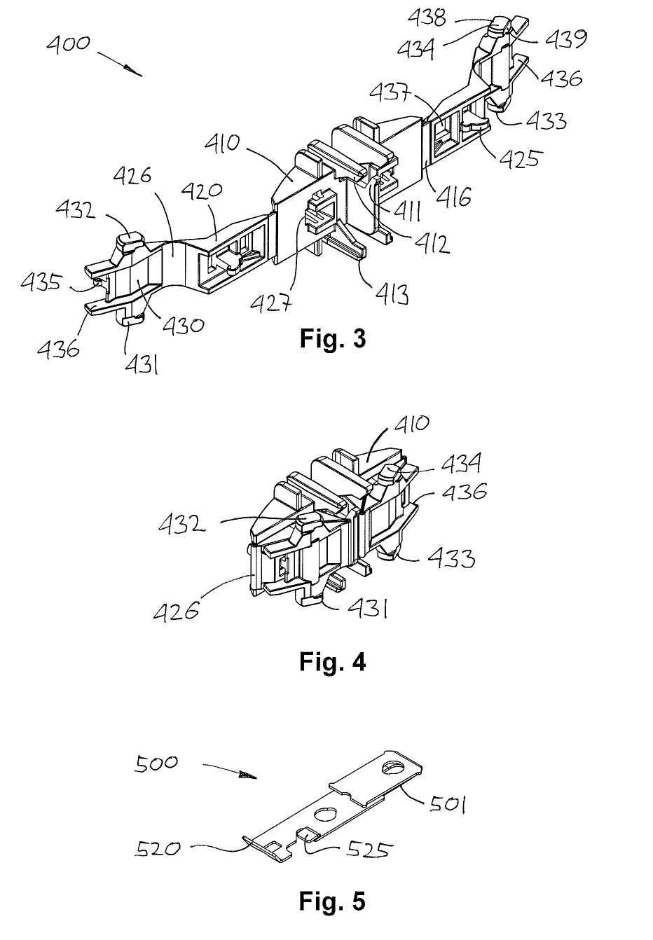

FIG. 3 shows an actuation member 400 which in the shown embodiment comprises a base member 410 with two symmetrically arranged accordion-like legs, each leg comprising an intermediate member 420 and a distal pivot member 430. Each pivot member has a free distal end portion 436 and a proximal portion connected to the intermediate member via a live hinge 426, the intermediate member being connected to the base member by a further live hinge 416, this allowing the actuation member to fold from a stretched configuration as in FIG. 3 to a folded configuration as in FIG. 4. Between the proximal and the distal portions the pivot members are provided with an axle having a first pair 431, 432 respectively a second pair 433, 434 of opposed axle heads adapted to engage the corresponding axle seats 231, 232 respectively 233, 234 to thereby form two pivoting joints. As appears, the generally circular axle heads are each provided with a pair of opposed flattened peripheral portions 438, 439 allowing the axle heads to be withdrawn from the axle seats via the peripheral openings 238 when correspondingly positioned, this as explained in greater detail below. The distal and intermediate elements are provided with locking means (here: hooks) 435, 425 adapted to releasably engage corresponding structures 437, 427 (here: openings) in the intermediate respectively the base member, this allowing the actuation member to be arranged and locked in the folded configuration seen in FIG. 4. The base member comprises mounting means (here: latches 415) adapted to engage corresponding mounting means (here: hooks, not shown) inside the cover 300. The base member is further provided with a pair of locking projections 412 and a release projection 411 for engaging the distal respectively the proximal surface of the slanted edge 520 of the locking member 500. Alternatively the actuation member may be formed integrally with the cover. Opposed to the locking projections 412 a pair of guide projections 413 are provided, whereby the two pair of projections serve to guide the actuation member into engagement with the case during assembly. The base member is further provided with four stop projections 414.

The flexible metal locking member 500 shown in FIG. 5 comprises in addition to the structures 520, 525 described above a U-shaped proximal end portion 501 allowing the locking member to be mounted in and locked to the case 200. In this position the distal end can flex inwardly relative to the case. Although the locking member in the shown embodiment is a separate element, the corresponding functional structures could also be formed integrally with the case.

Before turning to a description of the working principle of the disclosed embodiment of the invention, it will be described how the different components can be assembled in a cost-efficient way.

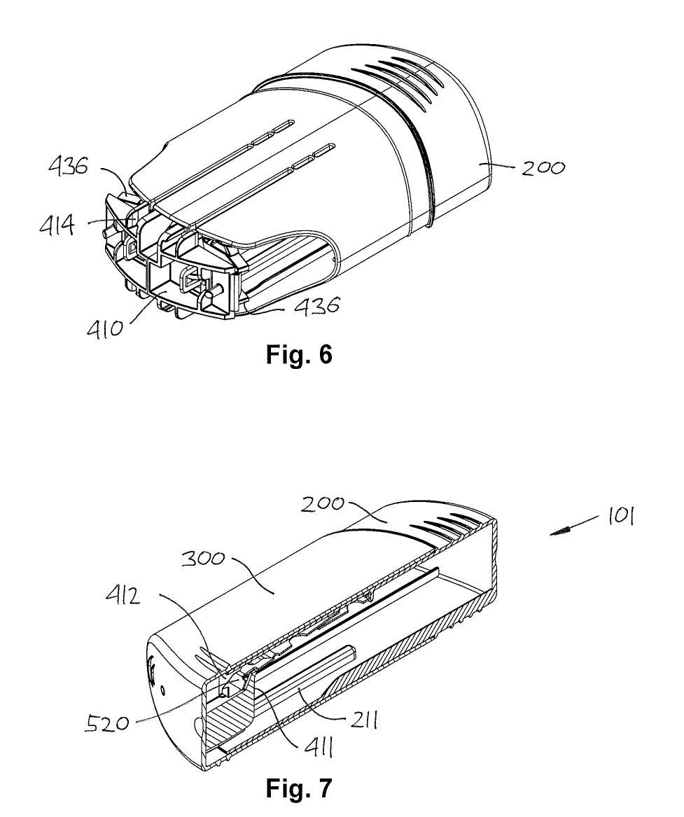

In a first step the locking member 500 is mounted in the case 200 where after the pump unit with the pump assembly in its initial position is placed in the case. In a next step the actuation member 400 in its folded configuration is mounted in the case whereby the axle heads are positioned in their corresponding axle seats. When the actuation member is in its folded configuration the axle heads cannot be inserted into the axle seats via the peripheral openings 238, however, the inherent flexibility of the case allows the axle seats of the case to “open and close” around the axle heads when the axle heads for forced into engagement with the ramps 239, this allowing the actuation member to be inserted in a general axial direction (as defined by the case). Indeed, alternatively the actuation member could be “folded into place” via the openings in the axle seats. During this insertion the release projection 411 first passes below the slanted edge 520 of the flexible locking member 500 where after the locking projections 412 engage the proximal surface of the slanted edge, thereby forcing the latch 525 into engagement with the indentation 125 in the pump assembly, this locking the pump assembly 120 relative to the case. At the same time a portion of the actuation member engages the free edge portion 112 of the pump unit housing, thereby effectively locking the pump assembly relative to the pump housing, this preventing undesired movement of the two components during handling of the assembly. It should be noted that in this position the distal free ends 436 of the pivot members 430 do not engage the pump assembly. To properly position the base member 410 relative to the case the two pairs of projections 412, 413 are adapted to fit into the gap between the interior ridges 211, just as the stop members 414 prevents the base members from being inserted too deep into the case by engaging the free distal edge of the case. FIG. 6 shows (with the pump unit removed) the assembly when the actuation member has been mounted in the housing.

In a final step the cover is slid over the case with the guide flanges 301 being inserted in the corresponding grooves 210 of the case. As the cover is mounted on the case the latches 415 of the actuation member engages the corresponding hooks on the interior end surface of the cover, thereby permanently locking the two components to each other. As the cover prevents the axle seats from moving outwards, this ensures that the actuation member is properly locked to the case via the pivoting joints. In the fully mounted position locking protrusions 315 on the interior of the cover releasably engages corresponding locking grooves 215 on the exterior surface of the case just as the circumferential seal structure 219 on the case effectively seals the gap between the cover and case, thereby protecting the interior of the container from e.g. dust and water. FIG. 7 shows (with the pump unit removed) the assembled product, especially how the projections 411, 412 are arranged on each side of the slanted edge 520.

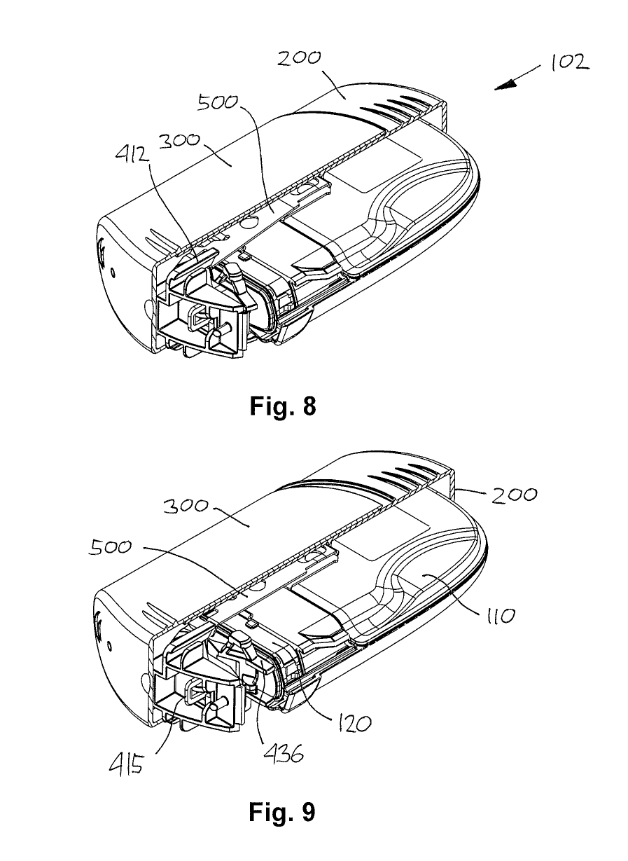

With reference to FIGS. 8-11 situation of use will be described in which the pump unit will be actuated as the cover is removed from the case.

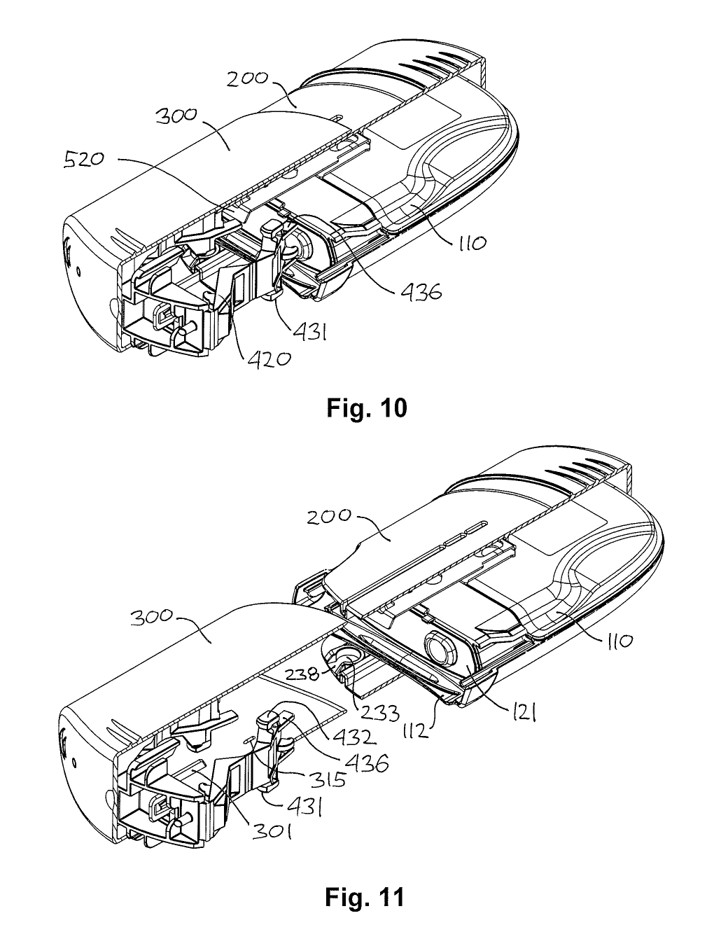

FIG. 8 shows the assembly 102 in its initial state as it may be supplied to a user. When the user starts to pull the cover and case from each other, thereby disengaging the case lock 315, 215, the pivot members start to rotate as the folded legs start to unfold, however, during the first few millimetres of cover movement relative to the case the distal free ends 436 of the pivot members 430 do not engage the pump assembly (see FIG. 9). During this initial movement the locking projections 412 disengage the proximal surface of the slanted edge, thereby allowing the biased free end of the flexible locking member 500 to return to its initial configuration, whereby the latch 525 is pulled out of engagement with the indentation 125 in the pump assembly, this un-locking the pump assembly 120 relative to the pump housing. In case the locking member do not return to its initial position, the release projection 411 will ensure that this happen when it is pulled against the proximal surface of the slanted edge 520, this forcing the latch 525 out of engagement with the indentation 125. The situation in which the locking member has disengaged the pump assembly and the pivot members have just touched the pump assembly is shown in FIG. 10.

As the cover is pulled further apart from the case the pivot members continue to rotate, the pivoting free ends 436 thereby forcing the pump assembly from its initial position towards its actuated position fully inserted into the pump housing. As appears, the two symmetrically arranged pivot members will ensure that the pump assembly is moved linearly into the pump housing.

When the pivot members have been rotated to fully insert the pump assembly into the pump housing as shown in FIG. 10, the pivot members have been rotated to a position in which the width of the axle heads (as seen from the axle seat openings) corresponds to the width of the openings, this allowing the axle heads to be pulled out of engagement with the axle seats, whereby the cover and the thereto attached actuation member can be fully removed from the case as seen in FIG. 11. The user can now easily grab the activated pump unit and remove it from the case.

The above-described pump unit may be adapted to be used in combination with a patch unit of the type shown in e.g. EP 1 527 792 or WO 2006/077263, which is hereby incorporated by reference. In WO 2006/077263 a pump unit is shown in which a pump assembly (comprising pump and actuator) is arranged stationary in a pump housing, the pump comprising a moveable needle connector which is pushed into contact with the reservoir when the pump unit is connected to a patch unit for the first time. Compared with this earlier version the above-described embodiment of a pump unit comprises a pump of the type shown in WO 2009/021950 (hereby incorporated by reference) where a needle connector projects from the pump, a fluid communication between the pump and reservoir being established by moving the pump (and thus the pump assembly) relative to the reservoir.

With reference to FIGS. 1-11 an embodiment of the present invention has been described in which a pump unit comprising a pump assembly slidingly arranged in a housing can be actuated during opening of a container, however, this is just an example illustrating the principle of the present invention. It should be clear that the invention advantageously can be used in combination with most devices in which a motion-based actuation of a device arranged in a container is relevant.

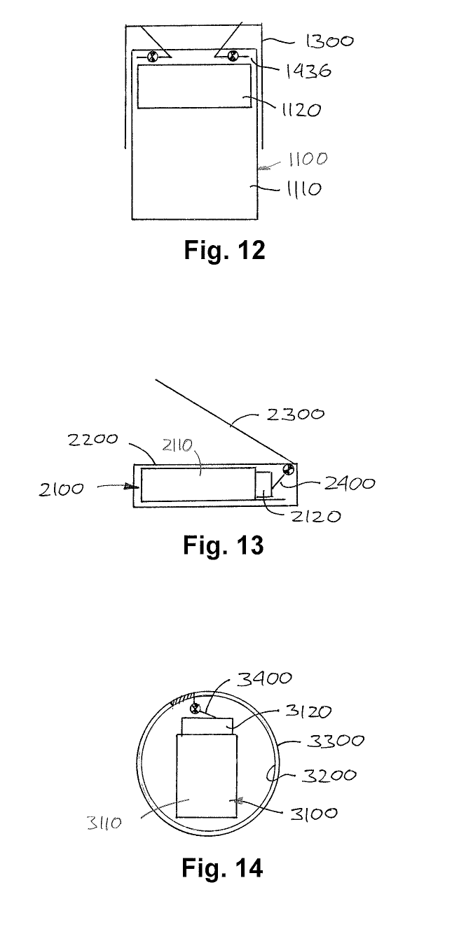

As also described above, a given device does not have to be fully enclosed in the interior of a container to implement the present invention, it may suffice if only a portion of a device is covered or enclosed with a member (e.g. protecting cover) which is to be removed prior to use. For example, FIG. 12 shows an embodiment in which a cover member 1300 directly engages pump housing 1110 of device 1100, this allowing pump unit 1120 to be moved into the housing. As appears, in this embodiment the pump housing carries the axle seats of the actuation member 1436.

FIG. 13 shows an alternative embodiment in which a device 2100 is arranged in a container comprising a box 2200 with a hinged lid 2300, the lid comprising an actuation member 2400 pushing a portion 2120 of the device into an actuated position relative to the device housing 2110. FIG. 14 shows a further alternative embodiment in which a device 3100 is arranged in a container comprising a circular box 3200 with a rotational lid 3300 attached by a bayonet coupling, the box comprising an actuation member 3400 pushing a portion 3120 of the device into an actuated position relative to the device housing 3110 when the lid is rotated to unlock the coupling.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. An assembly, comprising a device (100, 1100, 2100, 3100) and a cover (101, 1300, 2200, 3200) arranged to cover at least a portion of the device in an initial position, the device comprising:
a first portion (110, 1110, 2110, 3110), and
a second portion (120, 1120, 2120, 3120) moveable relative to the first portion between an initial and an actuated position,
wherein movement of at least a portion (300, 1300, 2300, 3300) of the cover from the initial position in a first direction results in the second portion of the device being moved in a second direction between the initial and the actuated position, the first direction being different from the second direction, and
wherein the force required to move the second portion of the device in the second direction is provided by movement of the at least a portion of the cover.

2. An assembly (102) as in claim 1, wherein the cover is in the form of a container (101) comprising:
a cavity portion (200) and a closure member (300) attached thereto and which together form an enclosure in which the device is arranged initially,
wherein movement of the closure member from an initial position in a first direction results in the second portion of the device being moved in a second direction between the initial and the actuated position, the first direction being different from the second direction.

3. An assembly as in claim 1, wherein the cover is in the form of a closure member (1300) attached to the device (1100) to cover a portion thereof,
wherein movement of the closure member from an initial position in a first direction results in the second portion of the device being moved in a second direction between the initial and the actuated position, the first direction being different from the second direction.

4. An assembly as in claim 2, comprising an actuation structure (400, 1436, 2400, 3400) which moves the second portion of the device in the second direction when the closure member is moved in the first direction.

5. An assembly as in claim 4,
wherein the actuation structure comprises a pivot member (430) with a proximal and a distal portion (436), and
wherein the pivot member initially is connected to a part of the assembly, other than the closure member or the second portion, by a pivoting joint (431, 432, 231, 232) located between the proximal and the distal portion,
whereby movement of the closure member from the initial position results in a pivoting movement of the distal portion of the pivot member, the pivoting movement moving the second portion of the device in the second direction.

6. An assembly as in claim 5, wherein the pivoting joint is adapted to disengage when the pivot member has moved the second portion of the device to its actuated position, this allowing the closure portion to be removed from the structure to which it initially was attached.

7. An assembly as in claim 6, wherein the closure portion comprises an outer shell (300) connected to the proximal portion of the pivot member (430) by a flexible connection, this allowing a pulling force to be transmitted to the pivot member when the closure portion is moved in the first direction.

8. An assembly as in claim 4, wherein the actuation structure comprises two pivoting members (430) arranged to pivot in opposite directions when the closure member is moved from the initial position.

9. An assembly as in claim 4, wherein the actuation structure is adapted to exert an essentially axial force on the second portion relative to a predetermined axial direction of movement between the first and second portion, this preventing kinking of the second portion relative to the first portion in respect of the predetermined direction of movement.

10. An assembly as in claim 1, wherein the first and second directions are essentially opposite to each other.

11. An assembly as in claim 2, wherein the first device portion is in the form a housing (110) with an opening (111) in which the second device portion (120) is slidably arranged.

12. An assembly as in claim 11, wherein the closure member in its initial position seals the opening.

13. An assembly as in claim 1, wherein the cover comprises locking means (525) for directly or indirectly locking the second portion relative to the first portion when the cover is in its initial configuration, the locking means being adapted to unlock when at least a portion of the cover is moved away from its initial position.

14. An assembly as in claim 1, wherein the first portion comprises a fluid-filled reservoir, and the second portion comprises a fluid conducting structure, the fluid conducting structure adapted to be arranged in fluid communication with the reservoir in the actuated position.

15. A method of arranging a device in a container, comprising the steps of:
providing a container comprising a cavity portion and a closure member which together form an enclosure,
providing a device adapted to be arranged at least partly in the cavity portion,
providing an actuation unit comprising a pivot member, the actuation unit being adapted to lock to the closure portion,
arranging the device at least partly in the cavity portion,
arranging the actuation unit with the pivot member in pivoting engagement with either the cavity portion or the device,
arranging the closure member in engagement with the cavity portion to thereby form an enclosure in which the device is arranged, and
locking the actuation unit to the cover portion.
